(19) United States
(12) Patent Application Publication
(10) Pub. No.: US 2024/0024220 A1
(43) Pub. Date: Jan. 25, 2024

(54) NEUROTOXIN COMPOSITIONS AND METHODS

(71) Applicant: YOUNGSUK YI, POTOMAC, MD (US)

(72) Inventor: YOUNGSUK YI, POTOMAC, MD (US)

(21) Appl. No.: 17/055,572

(22) PCT Filed: May 6, 2020
(86) PCT No.: PCT/US2020/031681

§ 371 Date: Nov. 14, 2020

Related U.S. Application Data
(60) Provisional application No. 62/846,621, filed on May 11, 2019.

(51) Int. Cl. A61K 8/64; C12N 5/0793; C07K 1/36

(52) U.S. Cl. CPC A61K 8/64; C12N 5/0619; C07K 1/36; C12N 2506/45

(57) ABSTRACT

Present invention provides improved compositions and methods of releasing cargo molecules to recipient cells. Described herein are engineered exosomes carrying neurotoxins and/or compounds, and releasing the neurotoxins to recipient cells by permeating the skin.

### CROSS-REFERENCE

This application claims the benefit of PCT Application No. PCT/US20/31681, filed on May 6, 2020, which claims the benefit of U.S. Provisional Application No. 62/846,621, filed May 11, 2019, which application is incorporated herein by reference in its entirety.

### BACKGROUND OF THE INVENTION

Neurotoxins are used for the treatment and prevention of various diseases as well as for cosmetic applications. Examples of neurotoxins include, but are not limited to, curare, bungarotoxin, saxitoxin, tetrodotoxin, tetanus toxin, or botulinum toxins. Curare neurotoxins are alkaloids that are the active ingredients of arrow poisons used by South American Indians. Curare alkoids have muscle relaxant properties because they block motor end plate transmission, acting as competitive antagonists for acetylcholine. Bungarotoxin is a neurotoxic protein derived from the venom of an elapid snake known as Bungarus multicinctus. Alpha-bungarotoxin blocks nicotinic acetylcholine receptors, while beta- and gamma-bungarotoxins act presynaptically causing acetylcholine release and depletion. Saxitoxin is a neurotoxin produced by the red tide dinoflagellates, Gonyaulax catenella and G. Tamarensis. Saxitoxin binds to sodium channels, thus blocking the passage of action potentials. This toxin was originally isolated from the clam, Saxidomus giganteus. Tetrodotoxin is a neurotoxin derived from the Japanese puffer fish. Tetrodotoxin also binds to sodium channel, and its activity somewhat resembles that of saxitoxin.

Tetanus toxin is a neurotoxin caused by the anaerobic, spore-forming bacillus Clostridium tetani. Clostridium tetani usually enters the body through contaminated puncture wounds although it may also enter through burns, surgical wounds, cutaneous ulcers, injection sites etc. Tetanus toxicity is often accompanied with sustained muscular contraction caused by repetitive nerve stimulation.

The tetanus neurotoxin acts mainly in the central nervous system, while botulinum neurotoxin acts at the neuromuscular junction; both act by inhibiting acetylcholine release from the axon of the affected neuron into the synapse, resulting in paralysis. The effect of intoxication on the affected neuron is long-lasting. The tetanus neurotoxin is known to exist in one immunologically distinct serotype.

Tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains.

The genus Clostridium has more than one hundred twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and shows a high affinity for cholinergic motor neurons. Symptoms of botulinum toxin intoxication can progress from difficulty walking, swallowing, and speaking to paralysis of the respiratory muscles and death. Botulinum toxins can cause neuroparalysis, or botulism, in mammals. There are at least seven known serotypes of botulinum toxins: toxins A, B, C₁, D, E, F, and G (“BoNT/A, /B, /C1, /D, /E and/F”).

The botulinum toxins are released as complexes comprising the 150 kD protein molecule along with associated non-toxin proteins. The BoNT/A complex can be produced as 900 kD, 500 kD and 300 kD forms. BoNT/B and C1 are produced as only a 500 kD complex. BoNT/D is produced as both 300 kD and 500 kD complexes. BoNT/E and F are produced as only approximately 300 kD complexes. The complexes are believed to contain a non-toxin hemaglutinin protein and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when toxin is ingested.

The clostridial neurotoxins are synthesized by the bacterium as a single polypeptide that is modified post-translationally to form two polypeptide chains joined together by a disulfide bond. The two chains are termed the heavy chain (H), which has a molecular mass of approximately 100 kDa, and the light chain (L), which has a molecular mass of approximately 50 kDa. The clostridial neurotoxins bind to an acceptor site on the cell membrane of the motor neuron at the neuromuscular junction and are internalized by an endocytosis. The internalized clostridial neurotoxins possess a highly specific zinc-dependent endopeptidase activity that hydrolyses a specific peptide bond in at least one of three proteins, synaptobrevin, syntaxin or SNAP-25, which are crucial components of the neurosecretory machinery. This enzymatic activity of the clostridial toxins results in a prolonged muscular paralysis. The zinc-dependent endopeptidase activity of clostridial neurotoxins is found to reside in the L-chain. The clostridial neurotoxins are highly selective for motor neurons due to the specific nature of the acceptor site on those neurons. The specific neuromuscular junction binding activity of clostridial neurotoxins is known to reside in the carboxy-terminal portion of the heavy chain component of the neurotoxin molecule, a region known as Hc.

BoNT/A is the most lethal natural biological agent known to man. The LD₅₀ in mice of purified BoNT/A is about 50 picograms. One unit (U) of botulinum toxin is defined as the LD₅₀ upon intraperitoneal injection into female Swiss Webster mice weighing 18-20 grams each. The different serotypes of botulinum toxin vary in the animal species that they affect and in the severity and duration of the paralysis they evoke. For example, it has been determined that BoNT/A is 500 times more potent, as measured by the rate of paralysis produced in the rat, than is botulinum toxin serotype B (BoNT/B). Additionally, botulinum toxin type B (“BoNT/B”) has been determined to be non-toxic in primates at a dose of 480 U/kg which is about 12 times the primate LD₅₀ for BoNT/A.

Regardless of serotype, the molecular mechanism of toxin intoxication appears to be similar and to involve at least three steps or stages. In the first step of the process, the toxin binds to the presynaptic membrane of the target neuron, e.g., cholinergic motor neuron, through a specific interaction between the H chain and a cell surface receptor; the receptor is thought to be different for each serotype of botulinum toxin and for tetanus toxin. The carboxyl end segment of the H chain, Hc, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This last step is thought to be mediated by the amino end segment of the H chain, HN, which triggers a conformational change of the toxin in response to a pH of about 5.5 or lower. Endosomes are known to possess a proton pump which decreases intra endosomal pH. The conformational shift exposes hydrophobic residues in the toxin, which permits the toxin to embed itself in the endosomal membrane. The toxin then translocates through the endosomal membrane into the cytosol.

The last step of the mechanism of botulinum toxin activity appears to involve reduction of the disulfide bond joining the H and L chain. The entire toxic activity of botulinum and tetanus toxins is contained in the L chain of the toxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. BoTN/A and E both cleave the 25 kD synaptosomal associated protein (SNAP-25), but they target different amino acid sequences within this protein. Tetanus neurotoxin, BoNT/B, D, F and G act on vesicle-associated protein (VAMP, also called synaptobrevin), with each serotype cleaving the protein at a different site. Finally, botulinum toxin serotype C1 (BoNT/C1) has been shown to cleave both syntaxin and SNAP-25. These differences in mechanism of action may affect the relative potency and/or duration of action of the various botulinum toxin serotypes. Each of these cleavages block the process of vesicle-membrane docking, thereby preventing exocytosis of vesicle content.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate.

BoNT/A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C1, D and E are synthesized by nonproteolytic strains and are therefore typically inactive when recovered from culture. Serotypes B and F are produced by both proteolytic and nonproteolytic strains and therefore can be recovered in either the active or inactive form. However, even the proteolytic strains that produce, for example, the BoNT/B serotype only cleave a portion of the toxin produced. The exact proportion of nicked to unnicked molecules depends on the length of incubation and the temperature of the culture. Therefore, a certain percentage of any preparation of, for example, the BoNT/B toxin is likely to be inactive, possibly accounting for the known significantly lower potency of BoNT/B as compared to BoNT/A. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy.

High quality crystalline botulinum toxin type A can be produced from the Hall A strain of Clostridium botulinum with characteristics of <3×10⁷ U/mg, an A260/A278 of less than 0.60 and a distinct pattern of banding on gel electrophoresis. Generally, the botulinum toxin type A complex can be isolated and purified from an anaerobic fermentation by cultivating Clostridium botulinum type A in a suitable medium. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative. The known process can also be used, upon separation out of the non-toxin proteins, to obtain pure botulinum toxins, such as, for example: purified botulinum toxin type A with an approximately 150 kD molecular weight with a specific potency of 1 −2×10⁸ LD₅₀ U/mg or greater; purified botulinum toxin type B with an approximately 156 kD molecular weight with a specific potency of 1 −2×10⁸ LD₅₀ U/mg or greater, and; purified botulinum toxin type F with an approximately 155 kD molecular weight with a specific potency of 1 −2×10⁷ LD₅₀ U/mg or greater.

Crystalline BOTOX® can be reconstituted in sterile normal saline without a preservative, (0.9% Sodium Chloride Injection). Botox can be denatured by bubbling or similar violent agitation.

The ability of Clostridial toxins, such as Botulinum neurotoxins (BoNTs), BoNT/A, BoNT/B, BoNT/C1, BoNT/D, BoNT/E, BoNT/F and BoNT/G, Tetanus neurotoxin (TeNT), Baratium neurotoxin (BaNT) and Butyricum neurotoxin (BuNT), to inhibit neuronal transmission are being exploited in a wide variety of therapeutic and cosmetic applications. Clostridial toxins commercially available as pharmaceutical compositions include, BoNT/A preparations. As an example, BoNT/A is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, strabismus, spasmodic dysphonia, strabismus and VII nerve disorder. BoNT/B has been approved by the FDA for the treatment of cervical dystonia. Clinical effects of peripheral intramuscular BoNT/A are usually seen within one week of injection. The typical duration of symptomatic relief from a single intramuscular injection of BoNt/A averages about three months.

For clinical use, it has been reported that BoNT/A has been administered as intramuscular injection as follows: (1) about 75-125 units of BOTOX® to treat cervical dystonia; (2) about 5-10 units to treat glabellar lines (brow furrows) (5 units injected intramuscularly into the procerus muscle and 10 units injected intramuscularly into each corrugator supercilii muscle); (3) about 30-80 units to treat constipation by injecting into the puborectalis muscle; (4) about 1-5 units to treat blepharospasm by injecting the lateral pre-tarsal orbicularis oculi muscle of the upper lid and the lateral pre-tarsal orbicularis oculi of the lower lid; (5) about 1-5 units to treat strabismus by injecting into the extraocular muscles; (6) about 25 units to treat migraine via pericranial injection (injected symmetrically into glabellar, frontalis and temporalis muscles); and (7) to treat upper limb spasticity following stroke by injecting into five different upper limb flexor muscles as follows: about 7.5 units to flexor digitorum profundus: 7.5 U to 30 U; about 7.5 to 30 units to flexor digitorum sublimus; about 10 to 40 units to flexor carpi ulnaris; about 15 to 60 units to flexor carpi radialis; and/or about 50 to 200 units to biceps brachii. It is also been suggested that a botulinum toxin can be used to weaken the chewing or biting muscle of the mouth so that self-inflicted wounds and resulting ulcers can heal; permit healing of benign cystic lesions or tumors; treat anal fissure; and treat certain types of atopic dermatitis. Additionally, it has been suggested that administering botulinum toxin to the foot to treat excessive foot sweating, spastic toes, idiopathic toe walking, and foot dystonia.

Exosomes are membrane-bound vesicles. They differ from other vesicles based on their biogenesis and biophysical properties, including size and surface protein markers. Exosomes are homogenous small particles ranging from 40 to 300 nm in size.

Some exosomes are derived from the endocytic recycling pathway. Generally, small, endocytic vesicles are formed via the fusion of the plasma membrane. As the vesicles mature, they become endosomes where intraluminal vesicles bud off into an intra-vesicular lumen, forming multi-vesicular bodies. They then directly fuse with the plasma membrane, and exosomes are released into the extracellular space.

The endosomal sorting complex required for transport (ESCRT complex) regulates the exosome biogenesis. Through the ESCRT-dependent or independent pathways, exosomes are formed and packaged within endosomes using sphingolipids and tetraspanins. Endosomes formed in this way are either digested by entering the lysosomal pathway, or fused with the plasma membrane and thereby releasing the exosomes within the endosomes. Exosomes contain a variety of biological material, including, but are not limited to, miRNAs, mRNAs, proteins such as Alix, Tsg101, I-integrin, MFG-E8, ICAM-1, the membrane markers such as CD63 and CD81, or a combination thereof. To date, about 134 studies on exosomes have reported 4,049 proteins, 1,639 mRNA molecules, 58 lipid molecules, and 764 miRNA molecules.

Exosomes function as signaling modalities, capable of modifying the phenotype of recipient cells. As a vehicle for delivering cargo molecules between cells, exosomes can directly mediate the transfer of biological material between cells. Exosomes can trigger a signal transduction pathway by providing a ligand to a molecule at cell surface, or change the molecular composition of the recipient cells.

Exosomes can function as a communication modality among different organs, as they can travel via the blood or cerebrospinal fluid. In the CNS, exosomes are involved in the crosstalk between neurons and glia. It has been reported that release of exosomes may affect synaptic activities by providing RNAs, proteins or lipids that function as signaling molecules. It has also been reported that exosomes are involved in the maintenance of myelination.

Exosomes secreted by neurons can deliver neuron-derived cargo specifically to other neuronal cells, such as astrocytes. Neuronal exosomes carrying miR-124a was found to be associated with enhanced expression of glutamate transporter-1 (GLT1), suggesting the exosome may influence glutamate uptake in the astrocytes by enhancing the expression of GLT1. Glial exosomes have been shown to be neurotrophic and neuroprotective. Astrocytes secretes exosomes in response to stress, and these exosomes have been shown to transfer pro-survival factors such as HSP70, Synapsin-I and LIF.

In addition, exosomes are involved in other cellular processes such as antigen presentation, cell death, angiogenesis, inflammation, and coagulation.

Virtually all types of cells produce and release exosomes. Depending on the cell type, exosomes vary significantly in size, the nature of cargo, and functions. In the CNS, neurons and oligodendrocytes secrete exosomes upon neuronal depolarization or release of glutamate, suggesting the release of exosomes are related to synaptic transmission.

Recent studies suggested that human mesenchymal stem cell (MSC)-derived exosomes activate several signaling pathways, which are conducive in wound healing and cell growth. The roles of exosomes that are derived from umbilical cord stem cells in cutaneous collagen synthesis and permeation have been reported. These exosomes have various growth factors associated with skin rejuvenation. Unlike liposomes, certain exosomes, e.g., umbilical cord stem cell derived exosomes, have been shown to pass through the skin, approaching outermost layer of the epidermis 3 hours after being applied to the skin, and gradually approaching the epidermis 18 hours after the application. As a result, increased expressions of collagen I and elastin have been observed.

Mesenchymal stem cells (MSCs) are multipotent stromal cells that can differentiate into a variety of cell types, including osteoblasts, chondrocytes, myocytes, and adipocytes. MSCs have a great capacity for self-renewal while maintaining their multipotency. The cultured MSCs also express on their surface CD73, CD90 and CD105, while lacking the expression of CD11b, CD14, CD19, CD34, CD45, CD79a and HLA-DR surface markers. MSCs do not differentiate into hematopoietic cells. The term MSCs encompasses multipotent cells derived from other non-marrow tissues, such as placenta umbilical cord blood, adipose tissue, adult muscle, corneal stroma, or the dental pulp of deciduous baby teeth. MSCs do not have the capacity to reconstitute an entire organ. The standard test to confirm multipotency is differentiation of the cells into osteoblasts, adipocytes and chondrocytes as well as myocytes and neurons.

Human bone marrow derived MSCs show fibroblast-like morphology. MSCs are characterized morphologically by a small cell body with a few cell processes that are long and thin. The cells, which are long and thin, are widely dispersed and the adjacent extracellular matrix is populated by a few reticular fibrils but is devoid of the other types of collagen fibrils.

Bone marrow is a source of MSCs. These bone marrow MSCs do not contribute to the formation of blood cells and so do not express the hematopoietic stem cell marker CD34. The youngest and most primitive MSCs may be obtained from umbilical cord tissue, namely Wharton's jelly and the umbilical cord blood. However MSCs are found in much higher concentration in the Wharton's jelly compared to cord blood, which is a rich source of hematopoietic stem cells. Adipose tissue is a rich source of MSCs. The developing tooth bud of the mandibular third molar is a rich source of MSCs. Dental-pulp MSCs are capable of producing hepatocytes. MSCs are present in amniotic fluid. As many as 1 in 100 cells collected during amniocentesis are pluripotent MSCs.

MSCs have an effect on innate and specific immune cells. MSCs produce many molecules having immunomodulatory effects. These include prostaglandin E2 (PGE2), nitric oxide, indolamin 2,3-dioxigenase (IDO), IL-6, and other surface markers—FasL, PD-L1/2. MSCs have an effect on macrophages, neutrophils, NK cells, mast cells and dendritic cells in innate immunity. Macrophage phenotype is divided into two groups: the M1 phenotype has a pro-inflammatory effect, and the M2 has anti-inflammatory effect. MSCs are able to migrate to the site of injury where they produce PGE2, which in turn polarize macrophages to the M2 phenotype, initiating the anti-inflammatory effect. Further, PGE2 inhibits the ability of mast cells to degranulate and produce TNF-alpha. Proliferation and cytotoxic activity of NK cells is inhibited by PGE2 and IDO. MSCs also reduce the expression of NK cell receptors—NKG2D, NKp44 and NKp30. MSCs inhibit respiratory flare and apoptosis of neutrophils by production of cytokines IL-6 and IL-8. Differentiation and expression of dendritic cell surface markers is inhibited by IL-6 and PGE2 of MSCs. The immunosuppressive effects of MSC also depend on IL-10. MSC expresses the adhesion molecules VCAM-1 and ICAM-1, which allow T-lymphocytes to adhere to their surface. Then MSC can affect them by molecules which have a short half-life and their effect is in the immediate vicinity of the cell. These include nitric oxide, PGE2, HGF, and activation of receptor PD-1. MSCs reduce T cell proliferation between G0 and G1 cell cycle phases and decrease the expression of IFN-gamma of Th1 cells while increasing the expression of IL-4 of Th2 cells. MSCs also inhibit the proliferation of B-lymphocytes between G0 and G1 cell cycle phases. MSCs can produce antimicrobial peptides (AMPs). These include human cathelicidin LL-37, beta-defensines, lipocalin 2, and hepcidin. MSCs effectively decrease number of colonies of both gram negative and gram positive bacteria by production of these AMPs. In addition, the same antimicrobial effect of the enzyme IDO produced by MSCs was found. MSCs in the body can be activated and mobilized if needed. However, the efficiency is low.

### SUMMARY OF THE INVENTION

Neurotoxins have been used to inhibit neuronal transmission in a wide variety of therapeutic and cosmetic applications. In these applications, injection, i.e., piercing the skin with a hypodermic needle attached to a syringe, has been the preferred mode of administering the neurotoxins. The present invention provides compositions and methods useful for delivering neurotoxins to recipient cells without an injection. In comparison to traditional delivery via a needle, injection-free delivery is painless, less-prone to opportunistic infections associated with the use of hypodermic needle, and causes less swelling and redness.

In one aspect, described herein is a composition comprising, exosomes derived from exosome-producing cells; and neurotoxins selected from the group consisting of botulinum toxin A, B, C₁, D, E, F, and G, and tetanus toxin, wherein said neurotoxins are encapsulated in said exosomes and said neurotoxins are non-full-length polypeptide containing the catalytic domain of said neurotoxins that is unable to be bound to said neurotoxins' natural receptors.

In one embodiment, said exosomes contain neuronal cell specific glycoprotein.

In one embodiment, said exosomes contain Synapsin I.

In one embodiment, said exosome-producing cells are neuronal cells, neuronal stem cells, neuronal stem cells derived from an induced pluripotent stem cell, or neuronal cells differentiated from an induced pluripotent stem cell.

In one embodiment, said exosome-producing cells are fibroblasts.

In one embodiment, said exosome-producing cells are human dermal microvascular endothelial cells.

In one embodiment, equal to or less than 30 percent of said exosomes's lumen is filled with said neurotoxins.

In one embodiment, said exosomes have a diameter of equal to or less than about 150 nm.

In one embodiment, said exosomes have a diameter of equal to or less than about 40 nm.

In one embodiment, said neurotoxins are derived from bacterial, yeast, insect, or mammalian cells transiently or stably expressing a full-length polypeptide from the cDNAs of said neurotoxins.

In one embodiment, said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

In one embodiment, said composition further comprise an emollient selected from the group consisting of cerotyl linoleate, diisoestearyl dimerate, diisopropyl dimerate, stearyl oleate, stearyl linoleate, cerotyl oleate, melissyl oleate, melissyl linoleate, and cerotyl eicosapentanoate.

In one embodiment, said composition further comprise water, disodium EDTA, soldium hyaluronate, glycerin, palmitoyl tripeptide, dextran, caproyl tetrapeptide, xanthan gum, cetostearyl alcohol, shea butter, jojoba oil, coconut oil, cetearyl ethylhexanoate, squalene, carbomer, pheoxyethanol, ethylhexylglcerin, NaOH, corundum powder, Asparagopsis armata extract, collagen, magnesium stearate, cellulose, lactose, mannitol, methyl cellulose, or a combination thereof.

In one embodiment, said composition further comprise a buffered solution selected from the group consisting of phosphate-buffered saline, Hanks balance salt solution, or saline.

In one embodiment, said composition is packaged in a roller, a ball-pointed liquid container, a liquid dispenser, a sprayer, a spatula, a dropper, a hand-operated pump, or a tube.

In one embodiment, the amount of said exosomes in a package is about 4 mg.

In one aspect, described herein is a method of preparing exosomes comprising, culturing induced pluripotent stem cells; differentiating said induced pluripotent stem cells to neuronal stem cells; and isolating exosomes wherein said isolating is ultracentrifugation, ultrafiltration, size-exclusion chromatography, immunoisolations, precipitation, or microfluidics-based isolation.

In one aspect, described herein is a method of preparing exosomes comprising, culturing exosome-producing cells and encapsulating neurotoxins wherein said exosome-producing cells are 1OT1/2, BALB/3T3, L-M, NB4, 1A3, NIE-1 15, NG108-15, NIH3T3, NCTC, Neuro-2A, PC12, GH1, GH3, C6, L2, CHO, OHO, 6E6, PK15, LLC-PK1, ST, ESK-4, CPAE, BT, FB2, SBAC, NBL-6, COS-1, COS-7, or VV-1, SH-SY5Y, SK-N-DZ, SK—N—Fl, SK—N—SH, BE(2)-C, HeLa, HEK 293, MCF-7, HepG2, HL-60, IMR-32, SW-13, OHP3, or CHPS and wherein said encapsulating is receptor-mediated endocytosis, passive diffusion, sonication, saponication, heating, emulsification, freezing and thawing, or use of solvents.

In one aspect, described here is a method of applying a neurotoxin to skin, comprising: preparing said skin by a method selected from the group consisting of cleaning, surfactant treatment, derma abrasion, exfoliation, chemical peel, laser resurfacing, hair removal, massaging, warming, and cleaning of the skin; and applying a composition comprising exosomes having a diameter of equal to or less than 40 nm, and neurotoxins.

In one embodiment, said surfactant is sodium lauryl sulfate, ammonium lauryl sulfate, disodium lauryl sulfosuccinate, cocoamphocarboxyglycinate, cocoamidopropyl betaine, or alpha-olefin sulfonate.

In one embodiment, said exosomes contain neuronal cell specific glycoprotein.

In one embodiment, said exosomes contain Synapsin I.

In one embodiment, said neurotoxins are selected from the group consisting of botulinum toxin A, B, C₁, D, E, F, and G, and tetanus toxin.

In one embodiment, said exosomes are derived from neuronal cells, neuronal stem cells, neuronal stem cells derived from an induced pluripotent stem cell, or neuronal cells differentiated from an induced pluripotent stem cell.

In one embodiment, said exosomes are derived from fibroblasts.

In one embodiment, said exosomes are derived from human dermal microvascular endothelial cells.

In one embodiment, said compositions are delivered to neuronal cells.

In one embodiment, said compositions are delivered to human fibroblasts.

In one embodiment, said compositions are delivered to endothelial cells.

In one embodiment, equal to or less than 30 percent of the lumen of said exosomes are filled with said neurotoxins.

In one embodiment, said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

In one embodiment, said composition further comprise an emollient selected from the group consisting of cerotyl linoleate, diisoestearyl dimerate, diisopropyl dimerate, stearyl oleate, stearyl linoleate, cerotyl oleate, melissyl oleate, melissyl linoleate, and cerotyl eicosapentanoate.

In one embodiment, said composition further comprise water, disodium EDTA, soldium hyaluronate, glycerin, palmitoyl tripeptide, dextran, caproyl tetrapeptide, xanthan gum, cetostearyl alcohol, shea butter, jojoba oil, coconut oil, cetearyl ethylhexanoate, squalene, carbomer, pheoxyethanol, ethylhexylglcerin, NaOH, corundum powder, Asparagopsis armata extract, collagen, magnesium stearate, cellulose, lactose, mannitol, methyl cellulose, or a combination thereof.

In one embodiment, said composition further comprise a buffered solution selected from the group consisting of phosphate-buffered saline, Hanks balance salt solution, or saline.

In one embodiment, said composition is packaged in a roller, a ball-pointed liquid container, a liquid dispenser, a sprayer, a spatula, a dropper, a hand-operated pump, or a tube.

In one embodiment, the amount of said exosomes in a package is about 4 mg.

Described herein is a method of selecting exosomes suitable for releasing neurotoxins to neuronal cells in human skin comprising: culturing exosome-secreting cells; modifying said exosome-secreting cells or exosomes secreted therefrom to carry one or more neurotoxins in said exosomes; culturing neuronal cells in the presence of said exosomes; selecting for exosomes of which said neurotoxins are found in said neuronal cells; optionally sorting said exosomes of step four in accordance to their sizes; applying said selected and optionally sorted exosomes to a donated human skin; and selecting for exosomes of which said neurotoxins are found in the dermis layer of said human skin.

In one embodiment, said culturing is a primary culture of cells derived from peripheral neuron.

In one embodiment, said modifying is selected from the group consisting of transfection, transduction, extrusion, sonication, diffusion, saponication, heating, emulsification, and freezing and thawing.

In one embodiment, said exosomes are isolated from said exosome-producing cells by a method selected from the group consisting of ultracentrifugation, ultrafiltration, size-exclusion chromatography, immunoisolations, precipitation, and microfluidics-based isolation.

In one embodiment, said sorting is selected from the group consisting of ultracentrifugation, ultrafiltration, size-exclusion chromatography, immunoisolations, precipitation, and microfluidics-based isolation.

In one embodiment, said applying is incubating said skin with said exosomes for 2, 4, 8, 12, or 24 hours.

In one embodiment, said selecting of step seven is performed by a method selected from the group consisting of immunostaining, fluorescent microscopy, and western blot.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates engineered exosomes of the present invention delivering neurotoxins to recipient cells. The engineered exosomes with neurotoxins 1 and/or free form neurotoxins 4 pass through the outer layers of the skin 2, and enter into the inner area including the dermis 3. The engineered exosomes, or free form neurotoxins reach the recipient cell 5 by diffusion and the neurotoxins are delivered to the recipient cells by membrane fusion, or endocytosis.

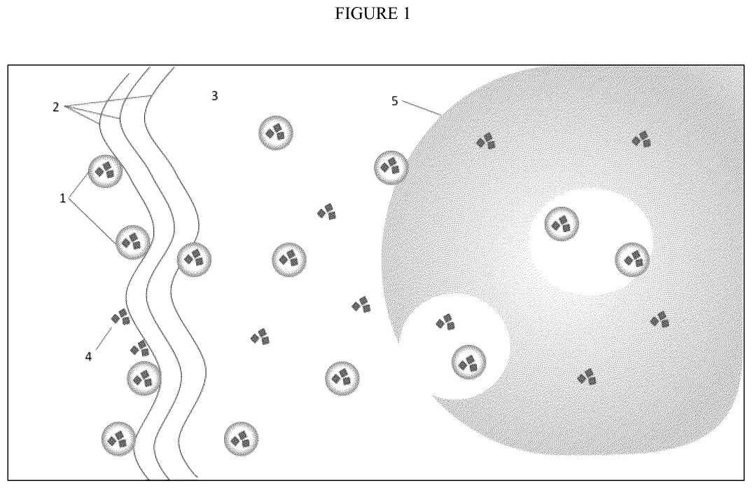

FIG. 2 illustrates a method of selecting exosomes suitable for releasing a neurotoxin to neuronal cells in human skin.

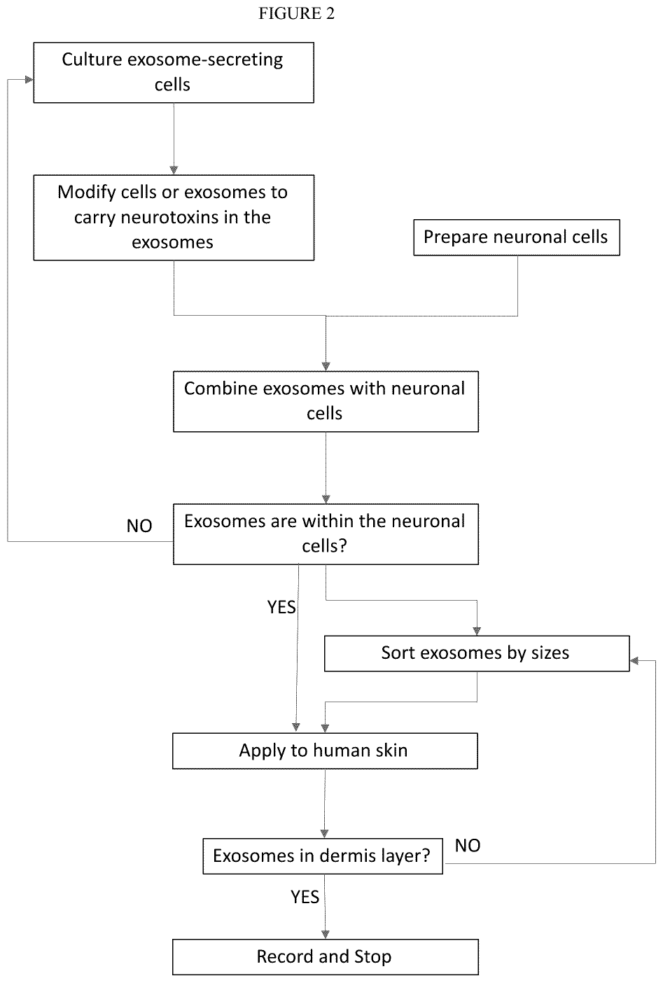

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions/Nomenclature

As used herein unless otherwise indicated, open terms such as “contain,” “containing,” “include,” “including,” and the like mean comprising.

Some embodiments herein contemplate numerical ranges. When a numerical range is provided, the range includes the range endpoints unless otherwise indicated. Unless otherwise indicated, numerical ranges include all values and subranges therein as if explicitly written out.

Some values herein are modified by the term “about.” In some instances, the term “about” in relation to a reference numerical value can include a range of values plus or minus 10% from that value. For example, the amount “about 10” can include amounts from 9 to 11. In other embodiments, the term “about” in relation to a reference numerical value can include a range of values plus or minus 10%, 9%, 8%, 7%, 6%, 5%, 4%, 3%, 2%, or 1% from that value.

As used herein, the article “a” means one or more unless explicitly stated otherwise.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “× g” is multiples of g, the standard acceleration due to gravity at the Earth's surface, “min” means minute(s), “C” means Celsius or degrees Celsius, as is clear from its usage, “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s)”, “uT” means microtesla, “g” means gram(s), “μg” or “ug” means microgram(s) and “ng” means nanogram(s), “% w/v” means weight/volume percent, “% v/v” means volume/volume percent, “HPLC” means high-performance liquid chromatography, “UPLC” means ultra-performance liquid chromatography, and “GC” means gas chromatography.

Compositions and methods described herein provide for engineered exosomes useful for carrying neurotoxins, chemical compound, or both, as a cargo. In accordance with one aspect of the invention, the engineered exosomes release the cargo by fusing with recipient cells. In another aspect, the engineered exosomes pass through the human skin via permeation.

Compositions and methods described herein have certain advantages over the current practice of injecting neurotoxins. Using engineered exosomes as a vehicle to deliver the cargo molecule, such as neurotoxins, provides safer and more cost effective than delivering purified neurotoxins. Direct injection of neurotoxins works by passive diffusion of the neurotoxins at the site of injection. The process exposes unintended target cells at the site of application to injected neurotoxins, leading to potentially causing side effects. In contrast, exosome mediated delivery is cell-type specific, and exosomes can be engineered to deliver the neurotoxins only to the intended target cells. Since exosomes are much bigger than neurotoxins such as BoNTs, passive diffusion plays a lessor effect in spreading the neurotoxin at the site of application.

The engineered exosomes of the present invention are manufactured by modifying the exosome-producing cells or by modifying naturally-occurring exosomes. Both methods require selecting one or more exosome-producing cells as the first step of the manufacturing process. Cells originated from any one of the three germ layers have been found to produce exosomes. Thus, virtually any type of cells are useful for the present invention as a source of exosome-producing cells.

Primary cells useful for the present invention can be derived from the endoderm, the mesoderm, or the ectoderm. Cells derived from the endoderm include, but are not limited to, cells of the respiratory system, the intestine, the liver, the gallbladder, the pancreas, the islets of Langerhans, the thyroid, or the hindgut. Cells derived from the mesoderm include, but are not limited to, cells of the osteochondroprogenitor cells, the muscle, the digestive systems, the renal system such as renal stem cells, the reproductive system, bloods cells, or cells from the circulatory system, such as endothelial cells. Cells derived from the ectoderm include, but are not limited to, epithelial cells, cells of the anterior pituitary, cells of the peripheral nervous system, cells of the neuroendocrine system, cell of the teeth, cell of the eyes, cells of the central nervous system, cells of the ependymal, or cells of the pineal gland.

More specifically, primary cells of the central and the peripheral nervous system can be a source of exosome-secreting cells. These cells include, but are not limited to, Schwann cells, satellite glial cells, oligodendrocytes, or astrocytes. The neurons as used herein include, but are not limited to, peripheral neurons, interneurons, pyramidal neurons, gabaergic neurons, dopaminergic neurons, serotoninergic neurons, glutamatergic neurons, motor neurons from the spinal cord, or inhibitory spinal neurons.

Stem/progenitor cells are a special type of primary cells capable of renewing itself and/or differentiating into various cell types. Stem/progenitor cells of various origins have been found to produce exosomes. Stem/progenitor cells useful for the present invention can be isolated from umbilical-cord, umbilical cord blood, peripheral blood, embryo, adipose tissue, bone-marrow, hair-follicle, hair follicle, or the subgranular zone (SGZ) of the dentate gyrus (DG) in the hippocampus and the subventricular zone (SVZ) in the forebrain. In one embodiment, stem cells useful for the present invention are mesenchymal stem cells (MSCs).

As to the neural stem/progenitor cells, the developing, as well as the adult central nervous system (CNS), contain multipotent cells with unlimited self-renewal referred to as neural stem/progenitor cells (NSCs). NSCs make a vital contribution to the formation of the CNS during development as they generate neurons, astrocytes, and oligodendrocytes, the three major cell types in the CNS. The primary NSCs are a subtype of radial glial cells that generate transit amplifying cells or intermediate progenitors through asymmetric cell division. The intermediate progenitors comprise the more restricted neuronal and glial progenitor cells displaying limited self-renewal and proliferative activity to produce differentiated neurons and glia. In the postnatal and adult periods, only a few areas in the CNS display active NSCs proficient in generating neurons, astrocytes, and oligodendrocytes. These regions, known as neurogenic regions, include the subgranular zone (SGZ) of the dentate gyrus (DG) in the hippocampus and the subventricular zone (SVZ) in the forebrain.

Another source of exosome-producing cells useful for the present invention is induced pluripotent stem cells (iPSCs). Forced expression of Oct3/4, Sox2 and Klf4 (as well as OCT3/4, SOX2 and KLF4) is sufficient to generate an induced pluripotent stem cell out of an adult somatic cell, such as a fibroblast. Also, the combination of Oct3/4, Sox2, c-Myc and Klf4 (as well as OCT3/4, SOX2, C-MYC) and KLF4 can be sufficient for the generation of a iPSC from an adult somatic cell. In addition, the combination of OCT3/4, SOX2, NANOG and LIN28 can be efficient for reprogramming. These genes are cloned into a retroviral vector and transgene-expressing viral particles or vectors, with which the somatic cell is co-transduced. Human skin fibroblasts can also be co-transduced with all four vectors e.g. via protein transduction or naked DNA. iPSCs may be obtained from any adult somatic cell. Exemplary somatic cells include peripheral blood Mononuclear Cells (PBMCs) from blood or fibroblasts, such as fibroblasts obtained from skin tissue biopsies.

iPSCs described herein can be derived from adult human cells. Examples of human cells include, but are not limited to, adult dermal fibroblasts, fetal FRC5 lung fibroblasts, or newborn BJ-1 foreskin fibroblasts. Systems useful for expressing the genes driving the reprogramming of adult cells to iPSCs include, but are not limited to, viral traduction methods using adenovirus, retrovirus, lentivirus, or sendai virus, or non-viral transfection methods using mRNA transfection, miRNA transfection, transfection of transposons, minicircle vectors, or episomal plasmids. Some material known to be useful for increasing the reprogramming efficiencies include, but are not limited to, hTERT, SV 40 large T antigen, a histone deacetylase inhibitor such as valproic acid or sodium butyrate, a MEK inhibitor such as PD0325901, a TGF-beta inhibitor such as A-82-01, SB43152, an epigenetic modifier such as vitamin C, a ROCK inhibitor such as thiazovivin, a PI3K inhibitor such as PS48, or a glycolysis promotor such as 5% oxygen. iPSCs described herein can be derived from human cells using one or more of the factors known to be useful for increasing the reprogramming efficiencies.

iPSCs can also be cultured to become NSCs. In one embodiment, the exosome-secreting cells useful for the engineered exosomes described herein are NSCs derived from iPSCs.

Cell lines are also known source of exosomes. Cell lines useful for the present invention can be derived from the primary cells and stem/progenitor cells described herein, or can be purchased from commercial providers, such as American Type Culture Collection (ATCC). Non-limiting examples of the established cell lines include Mus musculus cell line 1OT1/2, BALB/3T3, L-M, NB4, 1A3, NIE-1 15, NG108-15, NIH3T3, NCTC, or Neuro-2A, Rattus norvegicus cell line PC12, GH1, GH3, C6, or L2, Cricetulus griseus cell line CHO, OHO, or 6E6, Sus scrofa cell line PK15, LLC-PK1, ST, or ESK-4, Bos Taurus cell line CPAE, BT, FB2, or SBAC, Equus caballus cell line NBL-6, Cercopithecus aethiops cell line COS-1, COS-7, or VV-1, or Homo sapiens cell line SH-SY5Y, SK-N-DZ, SK—N—Fl, SK—N—SH, BE(2)-C, HeLa, HEK 293, MCF-7, HepG2, HL-60, IMR-32, SW-13, OHP3, or CHPS.

In embodiments utilizing primary cells as a source of exosome, the exosome-secreting cells are isolated from the tissue of origin, and cultured in vitro to secrete exosomes. A methods of isolating and culturing primary cells is provided in the examples herein.

Once an in vitro culture of exosome-secreting cells is established, the cells, exosomes, or both are modified to produce the engineered exosomes of the present invention.

In the present invention, the exosome-secreting cells are modified by genetic engineering methods including, but are not limited to, transfection, transduction, or a combination thereof. By these methods, the exosome-secreting cells are modified with plasmids to drive the expression of one or more cargo molecules, preferably eukaryotic codon optimized neurotoxins. The cells can also be modified by electroporation, or by the use of a transfection reagent, to load the neurotoxins directly to the cells. A method of modifying the exosome-secreting cells is provided in the examples herein.

The cDNA of a neurotoxin is codon-optimized to be expressed in exosome-secreting cells. The method of building a cDNA from genomic DNA, and optimizing a prokaryotic codon to a eukaryotic codon is well known to those of ordinary skill in the art. For example, GCA, GCC, GCG and GCU are the four synonymous codons that encode Alanine (Ala). While the most abundant Ala isoacceptor representative in C. botulinum recognizes the GCA codon, the bacterium Escherichia coli recognizes GCG, the yeast Pichia pastors recognizes GCT and most multicellular eukaryotes appear to recognize GCC. Optimization also includes modifying G+C content to match that of a eukaryotic cells, polynucleotide region (polyadenine, polyA; polythymidine, polyT; polyguanine, polyG; and polycytosine, polyC), or a combination thereof. Briefly, the codon-optimized cDNA of botulinum toxin A is transient expressed in a cell line according to methods known in the art. Non-limiting examples of plasmid carrying neurotoxin cDNA for eukaryotic expression include pQB125 vector (Obiogene), or pcDNATm6 vector (Invitrogen).

In the present invention, the neurotoxins can be derived from bacterial cells, yeast, or insect cells, and encapsulated into the exosome by methods described herein. cDNAs of recombinant neurotoxins can be expressed in bacterial cells such as E. coli using a suitable bacterial expression vector, such as commercially available pET series vectors or in insect cells such as Sf9 cell line using a suitable insect cell expression vector. The recombinant neurotoxins can be harvested by standard chromatography techniques.

In the present invention, the exosomes are modified by physical methods to encapsulate a cargo. Physical methods can be passive diffusion, sonication, saponication, heating, emulsification, freezing and thawing, use of solvents, or a combination thereof. Encapsulation methods are provided in the examples herein.

In one embodiment, the encapsulation method is passive diffusion. The cargo molecule is simply incubated with the exosomes, and the cargo molecule diffuses into the exosomes along the concentration gradient. A cargo molecule and exosome can be incubated in phosphate-buffered saline at 22° C. for about 5, 10, 15, 20, 25, or 30 min.

In one embodiment, the encapsulation method is sonication. Exosomes from donor cells are mixed with a cargo and subsequently sonicated by using a homogenizer probe. The mechanical shear force from the sonicator probe compromises the membrane integrity of the exosomes and allows the cargo to diffuse into the exosomes during this membrane deformation. The membrane integrity of the exosomes has been found to be restored within an hour when the exosomes are incubated at 37° C. Cargo molecules are not only encapsulated inside the exosomes but also attached to the outer layer of the membrane.

In one embodiment, the encapsulation method is extrusion. Exosomes from donor cells are mixed with a cargo, and the mixture is loaded into a syringe-based lipid extruder with 100-400 nm porous membranes under a controlled temperature. During the extrusion, the exosome membrane is disrupted and vigorously mixed with the cargo.

In one embodiment, the encapsulation method is freeze/thawing. The exosomes are incubated with the cargo at room temperature followed by rapid freezing at −80° C. or in liquid nitrogen, and then thawing at room temperature. The process is repeated at least 3 cycles to ensure encapsulation.

In one embodiment, the encapsulation method uses a membrane permeabilizer, such as saponin. Saponin is a surfactant molecule that can form complexes with cholesterol in cell membranes and generate pores, thus leading to an increase in membrane permeabilization.

Various methods of encapsulations as described herein provide exosomes in which the lumen of the exosoms is filled with a cargo in varying percentages. For example, 1%, 2%, 3%, 5%, 7%, 10%, 15%, 20%, 25%, 30%, 35%, 40%, 45%, 50%, 55%, 60%, 65%, 70%, 75%, 80%, 85% or 90% of the lumen can be filled with a cargo. The loading efficiency depends on the hydrophobicity of the cargo molecules.

In the present invention, the exosomes are modified by biological process to encapsulate a cargo. Biological methods can be receptor-mediated endocytosis. In one embodiment, neuronal cells expressing an acceptor molecule for BoNTs on cell surface are used to encapsulate the cargo. Examples of the receptors involved in the endocytosis of the cargo include synaptic vesicle protein 2 (SV2), vesicle associated membrane proteins (VAMPs), and SNARE proteins. In another embodiment, cells are transfected or transduced with plasmids carrying the cDNA of SV2, and clones expressing SV2 on the surface are isolated and used to encapsulate the cargo.

The engineered exosomes of the present invention carry one or more cargo molecules. The cargo molecules, as used herein, is a macromolecule or a compound not naturally found in the exosome. The cargo molecule can be a neurotoxin, a compound with a known pharmaceutical effect on human, or a combination thereof.

As a cargo molecule, a compound with a known pharmaceutical effect on human can be codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, salts thereof, complexes thereof, or mixtures of any of the foregoing.

As a cargo molecule, the neurotoxins described herein act intracellularly to inhibit neuronal transmission. These neurotoxins include, but are not limited to, the clostridial neurotoxin, such as botulinum neurotoxins (BoNTs), or tetanus neurotoxins (TeNTs). For example, the BoNTs include, but are not limited to, BoNT/A, BoNT/B, BoNT/C1, BoNT/D, BoNT/E, BoNT/F, BoNT/G, or a combination thereof. Other types of neurotoxins can also be used in the present invention, including, but are not limited to, diphtheria toxin (DTx), Pseudomonas aeruginosa exotoxin A, shiga toxin, ricin toxin, anthrax lethal toxin (lethal factor, LF), anthrax edema toxin (edema factor, EF), curare, bungarotoxin, saxitoxin or a combination thereof.

The neurotoxins described herein include a full-length polypeptide with or without post-translational modifications or a portion of the full-length polypeptide with or without post-translational modifications. A non-limiting example of the partial polypeptide is an engineered polypeptide that retains the light chain or the catalytic domain of the BoNTs but lacks the ability bind to an acceptor site on the cell membrane of the motor neuron.

In the present invention, the exosomes described herein may include a combination of full-length polypeptide and a non-full-length polypeptide. In one embodiment, the non-full-length polypeptide is the catalytic domain of the neurotoxin. In one embodiment, the catalytic domain of the neurotoxin is the light chain of BoNTs. Exosomes bearing varying ratios of full-length and non-full-length neurotoxins are used for biphasic delivery of the neurotoxins to the target cells. The ratio can be 10:90, 20:80, 30:70, 40:60, 50:50, 60:40, 70:30, 80:20, or 90:10 between the full-length and non-full-length polypeptides of the neurotoxins. The various ratios of full-length and non-full-length neurotoxins in the exosome can give rise to the release of neurotoxins in which about 10%, 20%, 30%, 40%, 50%, 60%, 70%, 80%, or 90% of the neurotoxins are released during the first phase and the rest of the neurotoxins are released during the second phase.

The engineered exosomes described herein can carry one or more protein molecules. The protein molecules include, but are not limited to, Alix, Contactin-2/TAG1, BLBP, CAD 65, CALB 1, CD-90, CD171, CD3, CD45, CD61, CD63, CD68, CD81, CD 171, CD9, CD1, Choline transporter, Contactin-2/TAG1, DARPP-32, diverse neuron or astrocyte adhesive proteins, Dopamine Transporter (DAT), Doublecortin, EMR1, GABA Transporters 1-3, GDNF, GLAST (EAAT), Glutamate Transporter, HES-1, Intemexin a, KA1 analogs, Laminin-1, Lhx1/5, LI NAM, LRP6 (excitatory), microglial CD18/11, MSR1, NAP 22, NCAM, Nestin, NeuN, NeuN, Notch 1, Parkin, Pcp2, RC2, RGS8, S100, SCIP analogs, Semaphorin ligands, Serotonin Transporter (SERT), Sox2, Synapsin I, TorsinA, Transferrin, TSG101, Tyro 3, VAMP, Vesicular Acetylcholine Transporter, Vesicular GABA Transporter, Vesicular Glutamate T 1-3, Zenon, or a combination thereof. Depending on the type of the exosome-secreting cells, These molecules can be naturally occurring in the exosomes, or can be artificially expressed regardless of the type of the exosome-secreting cells or the exosomes therefrom.

The engineered exosomes described herein can carry one or more microRNAs (miRNA). The miRNA includes, but is not limited to, miRNA9, miRNA335, miRNA153, miRNA21, miRNA210, miRNA34a, miRNA451, miRNA874, miRNA 124, miRNA125, miRNA 132, miRNA 134, miRNA138, miRNA106, miRNA128, miRNA 140, miRNA 146, miRNA 148, miRNA15, miRNA181, miRNA193, miRNA212, miRNA27, miRNA320, miRNA381, miRNA431, miRNA432, miRNA484, miRNA539, miRNA652, miRNA7, miRNA93, miRNA95, miRNA133, miRNA433, miRNA542, or a combination thereof. In some embodiments, exosomes derived from NSCs of the present invention carry miRNAs. These miRNAs include, but are not limited to, Homo sapiens (hsa)-miR-1246, hsa-miR-4488, hsa-miR-4508, hsa-miR-4492, and hsa-miR-4516. In one embodiment, exosomes described herein contain sufficient amount of miRNAs that could exert a biological effect in recipient cells. In one embodiment, said biological effect is regeneration, driven by miR-1246. In another embodiment, said biological effect is skeletal muscle regeneration driven by miR-4488. Depending on the type of the exosome-secreting cells, These nucleic acid molecules can be naturally occurring in the exosomes, or can be artificially expressed regardless of the type of the exosome-secreting cells or the exosomes therefrom.

The engineered exosomes described herein are less than 150 nm in diameter. For example, the exosome is about 10 nm, 20 nm, 30 nm, 40 nm, 50 nm, 60 nm, 70 nm, 80 nm, 90 nm, 100 nm, 110 nm, 120 nm, 130 nm, 140 nm, or 150 nm.

The engineered exosomes described herein comprise macromolecules encapsulated in lipid bilayer. The lipid bilayer can be about 5 nm thick, less than 5 nm or more than 5 nm thick. The lipid membrane bilayer surrounds a hydrophilic lumen in which various cargo molecules can be stored. The composition of the bilayer includes, but is not limited to, ceramide, cholesterol, sphingolipids, phosphoglycerides with long and saturated fatty-acyl chains, polysaccharide such as mannose, polylactosamine, alpha-2,6 sialic acid, N-linked glycans, or a combination thereof.

Exosomes engineered as described above are isolated from exosome-secreting cells. Described herein are methods to isolate the engineered exosomes, and then sort the exosomes according to their physical properties. In one embodiment, the physical property is the diameter of the exosomes.

Methods useful for separating the engineered exosomes include, but are not limited to, ultracentrifugation, ultrafiltration, size-exclusion chromatography (SEC), immunoisolations, precipitation, microfluidics-based isolation, or a combination thereof. Ultracentrifugation as used herein is a centrifugation process optimized for generating exceptionally high centrifugal forces up to 1,000,000×g. Ultracentrifugation includes differential centrifugation, isopycnic ultracentrifugation, or moving-zone ultracentrifugation. Ultrafiltration as used herein is a size-based separation including the use of nanomembrane concentrators, syringe filter-based fractionation, or sequential filtration. Immunoisolation as used herein utilizes high-affinity antibodies conjugated to a solid surface such as glass or silica or magnetic particles. Precipitation as used herein relies on altering the solubility or dispersibility of exosomes in water-excluding polymers such as polyethylene glycol (PEG). Microfluidics-based isolation as used herein utilizes fabricated microfluidic devices employing various physical and chemical characteristics of exosomes including size, density, immunoaffinity, acoustic, electrophoretic, electromagnetic properties, or a combinations thereof. Methods of isolating and sorting the engineered exosomes are provided in the examples herein.

The engineered exosomes of the present invention release its cargo to recipient cells. The release is mediated by the fusion of the engineered exosomes with the recipient cells. The fusion can be non-specific wherein the engineered exosomes fuse with any type of cells, or cell-type specific wherein the engineered exosomes fuse with a particular type of cells. In one embodiment, the engineered exosomes release its cargo by fusing with human fibroblasts and peripheral neuron. In another embodiment, the engineered exosomes release its cargo by fusing with either human fibroblasts or peripheral neuron. In another embodiment, the recipient cells of the engineered exosomes can be epithelial cells, muscle cells, adipocytes, endothelial cells, or chondrocytes.

The cell-type specific tropism toward recipient cells can result in delivering substantially all of the cargo to only one type of cells, or in delivering about 5%, 10%, 20%, 30%, 40%, 50%, 60%, or 70% to one type of cells, and the rest are either undelivered or delivered to another cell type.

The cell-type specific tropism of the engineered exosome can be influenced by the type of the exosome-secreting cells. It can also be influenced by the molecules on the surface of an exosome. The surface molecules can be a naturally occurring molecules or artificially expressed molecules.

Exosomes of neuronal origins are particularly useful for the present invention, as these exosomes are known to deliver substantially all of its cargo to neuronal cells. As a long-distance, cell-type specific cell-to-cell communication modality, exosomes budding off from neuronal cells in one location travel to neuronal cells in another location, and deliver its cargo by selectively fusing itself to neuronal cells. Molecules such as Synapsin I is correlated with cell-type specific communications via exosome. Synapsin I is found on the surface of exosomes released from neuronal cells, including glial cells, but not in non-neuronal cells. In one embodiment, the engineered exosomes of the present invention has Synapsin I on the surface of the exosome. In one embodiment, Synapsin I-containing exosomes are derived from glial cells. In another embodiment, Synapsin I-containing exosomes are derived from cultured cortical astrocytes. In another embodiment, Synapsin I is expressed in non-neuronal exosome-secreting cells by means described herein.

Recipient cells as used herein can also be non-neuronal cells. Neurotoxins are known to trigger cellular responses in fibroblasts. For example, the expression profile of long noncoding RNAs (lncRNAs) in human dermal fibroblasts change dramatically when the fibroblasts are cultured in the presence of botulinum type A toxin at a range of 2.5 units/10⁶ cells. In addition to the lncRNAs, the level of certain mRNAs (NOS2, C13orf15, FOS, FCN2, SPINT1, PLAC8, BIRC5, NOS2, and COL19A1) are reduced. Culturing human monocytes with botulinum type D toxin results in the inhibition of tumor necrosis factor from the monocytes. Type A botulinum toxin treated human endothelial cells are protected from hypoxia-related injuries to the cells. For example, type A botulinum toxin attenuates hypoxia-triggered apoptosis in human dermal microvascular endothelial cells.

The engineered exosomes described herein are designed to reach epidermis or dermis when applied to human skin. In one embodiment, the exosomes are selected by one or more size exclusion methods described herein to be about 100 nm or less in diameter. In one embodiment, the exosomes are isolated from human umbilical cord blood derived mesenchymal stem cells, glial cells, iPSCs or NSCs wherein said exosomes are less than or equal to 100 nm in diameter.

The engineered exosomes described herein are delivered to recipient cells via permeation through the skin. The permeation can be driven by passive diffusion, or pre-treating the skin for permeation. Passive diffusion is facilitated by selecting exosomes according to their size, measured in diameter, and adding exosomes of certain size in a hydrophilic solution, such as phosphate-buffered saline or Hanks balanced salt solution, and directly applying the solution to the skin. Non-limiting examples of exosome sizes useful for passive permeation are about 10 nm, 20 nm, 30 nm, 40 nm, 50 nm, 60 nm, 70 nm, 80 nm, 90 nm, 100 nm, 110 nm, 120 nm, 130 nm, 140 nm, or 150 nm.

In some embodiments, the skin is pre-treated with surfactants prior to applying exosome-containing solution of the present invention. Non-limiting examples of surfactants include sodium lauryl sulfate, ammonium lauryl sulfate, disodium lauryl sulfosuccinate, cocoamphocarboxyglycinate, cocoamidopropyl betaine, alpha-olefin sulfonate, or a combination thereof.

In some embodiments, the skin is pre-treated by physical means. These physical methods are known to those of ordinary skill in the art. Non-limiting examples of physical pre-treatment include cleaning, surfactant treatment, derma abrasion, exfoliation, chemical peel, laser resurfacing, hair removal, massaging, warming, micro-needling, laser peel, or a combination thereof.

The engineered exosomes described herein are formulated in a medium suitable for applying to human skin. In some embodiments, the medium contains emollients. An emollient as used herein is esterification product of an aliphatic alcohol having at least 25 carbon atoms and an unsaturated aliphatic fatty acid having at least 16 carbon atoms. Non-limiting examples of emollients are cerotyl linoleate, diisoestearyl dimerate, diisopropyl dimerate, stearyl oleate, stearyl linoleate, cerotyl oleate, melissyl oleate, melissyl linoleate, or cerotyl eicosapentanoate.

In some embodiments, the medium is a buffered solution, such as phosphate-buffered saline, Hanks balance salt solution, or saline. Optionally, the solution may contain cosmetically acceptable preservatives.

In some embodiments, the engineered exosomes described herein are formulated as a cosmetic product, such as a balm, spray, gel, cream, serum, or a toner. Typical ingredients forming these various cosmetic base are known in the art.

In some embodiment, the cosmetic formulation used herein is compatible with an ingredient covered with lipid membrane, such as exosome, without negatively affecting the integrity of the lipid membrane structure. Non-limiting examples of compatible cosmetic ingredient include water, disodium EDTA, sodium hyaluronate, glycerin, palmitoyl tripeptide, dextran, caproyl tetrapeptide, xanthan gum, cetostearyl alcohol, shea butter, jojoba oil, coconut oil, cetearyl ethylhexanoate, squalene, carbomer, pheoxyethanol, ethylhexylglcerin, NaOH, corundum powder, Asparagopsis armata extract, collagen, magnesium stearate, cellulose, lactose, mannitol, methyl cellulose, or a combination thereof.

The passive nature of exosomes fusing to the plasma membranes of the recipient cells can result in delayed release of its cargo. In some embodiments, the engineered exosomes described herein release only a fraction of its total cargo within the first month of the application. Non-limiting examples of the fraction can be 1%, 2%, 4%, 8%, 16%, 32%, 50%, 70%, or 90% of the total cargo.

In some embodiments, the formulations of the present invention comprise liquid containing free form neurotoxins and engineered exosomes containing neurotoxins. As used herein, free form neurotoxins refer to neurotoxins not encapsulated within the exosomes. These formulations are useful for generating a biphasic delivery of neurotoxins wherein the two forms, i.e., the free form and the encapsulated form, have different rates of release. Non-limiting examples of the amount of the free form in the formulation can be 10%, 20%, 30%, 40%, 60% or 70%.

In some embodiments, the release of the cargo is biphasic wherein certain percentage of the cargo is rapidly released upon application of the formulation (“first phase”), and the rest of the cargo is released slowly over a long period of time (“second phase”). Non-limiting examples of the rapid release include about 10%, 20%, 30%, 40%, 60% or 70% of the cargo being released within the first phase. Non-limiting examples of the delayed release over a long period of time include releasing about 10%, 20%, 30%, 40%, 50%, 60%, or 70% of the cargo over about 3 months, 4 months, 5 months, 6 months, 7 months, 8 months, 9 months, 10 months, 11 months, or 12 months.

In some embodiments, the formulations containing the engineered exosomes described herein are housed in a device capable of delivering the exosomes to the skin. Non-limiting examples of the mechanical devices include a roller, a ball-pointed liquid container, a liquid dispenser, a sprayer, a spatula, a dropper, a hand-operated pump, a tube, or a combination thereof. A particular design of the device and specification thereof depends on the amount, frequency, and the area of the delivery of the formulations. A variety of designs capable of holding and delivering formulation containing liposomes are known in the art, and these device designs are compatible with the exosome-containing formulations of the present invention.

The engineered exosomes of the present invention can be formulated as low or high dosage forms. Low dosage forms may range from, without limitation, 1-50 micrograms per kilogram, while high dosage forms may range from, without limitation, 51-1000 micrograms per kilogram. In some embodiments, the engineered exosomes of the present invention are formulated as 3 mg, 4 mg, 5 mg, 6 mg, 7 mg, 8 mg, 9 mg, 10 mg, 50 mg, or 80 mg per package.

The compositions described herein can be administered repeatedly, including two, three, four, five or more administrations. In some instances, the compositions may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the purpose of the administration. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ.

Described herein is a method of selecting engineered exosomes suitable for releasing neurotoxins to the peripheral neurons of the skin via permeation.

To perform the selection, exosome-secreting cells are cultured according to methods described herein. The exosome-secreting cells are then genetically modified to express neurotoxins in inactive forms. The neurotoxin-containing exosomes are isolated according to methods described herein. Alternatively, unmodified exosomes are harvested from the exosome-secreting cells, and the exosomes are loaded with neurotoxins according to methods described herein. A culture of neuronal cells originated from the peripheral nerves is established. The isolated exosomes are added to the culture of neuronal cells, and incubated under standard cell culture conditions for a pre-determined time, such as 30 min, 1 hour, 2 hours, 4 hours, 8 hours, 16 hours, 24 hours, 48 hours, or 72 hours. The cells are harvested, washed, and the presence of neurotoxins in the neuronal cells are examined by methods known in the art, including Western blot, confocal microscopy, immunoassays using antibodies against the neurotoxin, or a combination thereof.

For those exosomes positively identified as capable of releasing neurotoxins to the neuronal cells, a further selection is conducted as follows. The exosomes are sorted according to their sizes by size-based separation methods described herein. A skin sample is prepared from donated human skin. The exosomes are then applied to the donated skin in accordance with their sizes. The exosomes and the skin are incubated for 2, 4, 8, 12, or 24 hours. The skin is then frozen and cryo-sectioned into slices. The slices are then immunostained with antibodies against the neurotoxin, and visualized by standard fluorescent microscopy method to locate the positions of neurotoxins in relation to the different layers of the skin. To visualize the locations of the exosomes, the slide is also immunostained with antibodies against surface molecules known to be present on the exosomes. Based on the result, an engineered exosome capable of passing through the skin's outermost layer is identified, and their size, the type of exosome-secreting cells, and the method of loading the neurotoxins are recorded for scale-up production.

In one embodiment, donated human skin is obtained from operations on breast reduction and abdominoplasty.

In one embodiment, the positively identified exosomes are diluted in PBS, and applied to the donated skin.

In one embodiment, Raster image correlation spectroscopy (RICS) microscopy is used to measure the permeability of the exosomes through various layers of the skin. Setting up fluorescent microscope for RICS measurements, such as the type of laser, excitation wavelength, bandpass filter settings, detector settings, are depending on the type of fluorophore conjugated with the antibodies used to immunostain the samples. In another embodiment, stimulated emission depletion (STED) microscopy is used to visualize the layers of skin.

### Example 1

Isolating Exosome-Secreting Neuronal Cells

Tissues containing primary cortical neurons are harvested from the brain, and washed with phosphate-buffered saline containing 1.5% glucose. The tissue is dissociated by passing through a 21 gauge needle. The cells are then separated from tissue debris by allowing the tissue debris to settle in a tube, and by transferring the supernatant to a new tube. To collect the cells, the supernatant is spun down for 5 min at 134×g. The collected cells are resuspended in a culture medium, such as commercially available Neurobasal media (Invitrogen™), supplemented with 2% B27 (Invitrogen™), 2 mM glutamine, 1.5% glucose, 100 ug/ml streptomycin and 60 ug/ml penicillin, and plated at a density 0.5×10⁶ cells per well in a 24-well plate precoated with 10 ug/ml poly-D-lysine. The culture is grown at 37° C. in a humidified atmosphere of 95% air and 5% CO₂. The neuronal identity of the culture can be confirmed by beta-tubulin III staining. After 7 days, the cells can be plated onto 5 ug/ml poly-D-lysine coated 100 mm dishes at a density of 6×10⁶ cells/dish.

### Example 2

Isolating Primary Astrocytes Secreting Exosomes

From the brain tissue, the meninges are discarded. Then the cortices are minced, which is followed by the dissociation of the by trituration through a 2 ml pipet. The dissociated tissue is incubated with trypsin to release the cells. The cells are then seeded in 10 cm plates in DMEM medium containing 10% fetal calf serum and 1% penicillin/streptomycin.

### Example 3

Isolating NSCs from the Brain

The SVZ and DC are dissected as follows: 1) place the rostral portion of the brain so that the cut coronal surface faces upwards and focus the microscope onto a higher magnification, 2) remove and discard the septum using fine curved forceps, 3) dissect the SVZ (the thin layer of tissue surrounding the ventricle) by placing the tip of one blade of a pair of fine curved forceps in the lateral corner of the lateral ventricle immediately under the corpus callosum and the other approximately 1 mm into the tissue immediately adjacent to the ventricle, 4) press down the forceps towards the base of the dish and towards the ventral aspect of the ventricle to remove a small triangular piece of tissue, and 5) place the dissected SVZ into a Petri dish on ice. To dissect the DG, 1) place the caudal portion of the brain in the Petri dish and cut along the longitudinal fissure using a scalpel, 2) under a dissection microscope, remove the cerebellum and the diencephalon using forceps, 3) refocus the microscope so that the borders around the DG are now visible, 4) to remove the dentate gyrus, insert the tip of a 27 G needle and slide along the border between the DG and Ammon's horn, and 4) using the fine forceps, free the DG from the surrounding tissue. Once SVZ is dissected, mince the tissue using a scalpel until no large pieces remain. The minced tissue is treated with 0.05% Trypsin-EDTA for 7 min at 37° C. The trypsin-digested tissue is centrifugated at 300 g for 5 min to pellet the cells. The cells are resuspended in growth medium comprising Neuralbasal media supplemented with 2% B27, 2 mM glutamine, 2 ug/ml heparin, and 50 units of penicillin/streptomycin, 20 ng/ml of epidermal growth factor, and 20 ng/ml of fibroblast growth factor. For dissected DG tissue, mince the tissue until no large pieces remain, and treat the tissue with 2.5 units/ml Papain, 1 unit/ml dispase, 250 units/ml DNase I for 20 min at 37° C. The digested tissue is then centrifuged at 130×g for 5 min to pellet the cells, and resuspended in 1 ml buffer solution ((1× HBSS, 30 mM Glucose, 2 mM HEPES (pH 7.4), 26 mM NaHCO₃), and centrifuged again. The NSCs from DG are then isolated as follows: 1) remove supernatant and resuspend the pellet in 5 ml of 20% Percoll. (To prepare 90% Percoll, add 4.5 ml of 100% Percoll to 0.5 ml of 10× PBS then further dilute this to 20% by adding 1.1 ml 90% Percoll to 3.9 ml 1× PBS), 2) centrifuge 450× g for 15 min, 3) remove the supernatant and resuspend the pellet in 10 ml buffer, 4) centrifuge at 130× g for 5 min, and 5) resuspend the pellet in 200 ul growth medium.

### Example 4

Culturing iPSCs and Deriving NSCs from the iPSCs

iPSCs are grown on a feeder layer made of irradiated mouse embryonic fibroblasts (iMEF) or on a coated plate such as Matrigel (BD), Cellstart (Invitrogen) or Vitronectin XF (Stem Cell Technologies) as feeder-free culture. For feeder layer, about 200,000 cells are plated in a well of a 6-well plate. For passaging of the cells, mechanical methods, such as colony picking or an enzymatic method can be used. Enzymes suitable for passing iPSC colonies are Collagenase type IV diluted with DMEM-F12, or Dispase. iPSC are detached from plates using Dispase (1 mg/ml) for 30 min at 37° C. with the help of a scraper. To form embryonic bodies, colonies are collected by sedimentation and resuspended in a medium comprising Knockout™ DMEM, Knockout™ SR, non-essential amino acids, penicillin/streptomycin, β-mercaptoethanol. The medium is supplemented with 10 MSB-431542 (Ascent Scientific), 1 μM dorsomorphin (Tocris), 3 μM CHIR 99021 (Axon Medchem), which is a GSK-3 inhibitor, and 0.5 μM purmorphamine (Alexis, also known as PMA or 2-(1-Naphthoxy)-6-(4-morpholinoanilino)-9-cyclohexylpurine), which is a Sonig Hedgehog signaling and WNT-signaling activator. 48 hours later, the medium is replaced by N2B27 medium comprising DMEM-F12 and Neurobasal medium mixed at 50:50 ratio, and supplemented with 1:200 N2 supplement (Invitrogen), 1:100 B27 supplement lacking vitamin A (Invitrogen), penicillin/streptomycin and glutamine, supplemented with 10 μM SB-431542, 1 μM dorsomorphin, 3 μM CHIR 99021 and 0.5 μM PMA. After additional 48 hours, the medium is replaced by N2B27 medium supplemented with 3 μM CHIR 99021, 0.5 μM PMA and 150 μM ascorbic acid (Sigma Aldrich), which is an antioxidant. Two days later, neural tube like structures are dissociated by pipetting, and small pieces are plated on Matrigel (BD Biosciences) coated 12-well plates in N2B27 medium supplemented with 3 μM CHIR 99021, 0.5 M PMA and 150 μM ascorbic acid. At day 8, the medium is exchanged by N2B27 medium supplemented with 3 μM CHIR 99021, 0.5 μM PMA, 150 μM ascorbic acid and 20 ng/ml basic fibroblast growth factor 2 (bFGF2). At day 12, cells are detached using dispase and cultivated in a medium on Matrigel coated 10 cm dishes comprising DMEM HAM's F12 medium supplemented with 200 ng/ml epidermal growth factor, 200 ng/ml bFGF2, N2 supplement, B27 supplement with vitamin A, glutamine, penicillin/Streptomycin and 150 unit/ml of human leukocyte inhibitory factor (LIF).

### Example 5

Differential Ultrafiltration of Exosomes

A mixture of live cells, dead cells and debris, culture medium, and exosomes are placed in a tube and centrifuged at 300×g for 10 min. The live cells are discarded, and the supernatant containing the rest are centrifuged at 2,000×g for 10 min. The dead cells are discarded and the remaining supernatant is centrifuged at 10,000×g for 30 min to remove cell debris. The supernatant, which contains exosomes and culture medium, is centrifuged at about 100,000-200,000×g for 70 min to pellet the exosomes. The supernatant, which contains culture medium, is discarded, and the exosome pellet is resuspended in a suitable solution, such as phosphate buffered saline, and re-centrifuged at about 100,000-200,000×g for 70 min.

### Example 6

Isopycnic Ultracentrifugation of Exosomes

A density gradient medium encompassing the entire range of densities of solutes in a sample is loaded to a centrifuge tube. After removing the cell debris, the supernatant is placed on a density gradient, and centrifuged at about 100,000-200,000×g, which results in exosome forming a narrow band suitable for extraction. A density medium includes, but is not limited to, cesium chloride. In this method, exosomes are extracted from the density region between 1.10 and 1.21 g/ml. Once the band containing exosomes is extracted, it is subjected to a brief centrifugation at about 100,000×g to further purify the exosomes.

### Example 7

Moving-Zone Ultracentrifugation of Exosomes

A sample containing exosomes is loaded as a thin zone on top of a gradient density medium of having a lower density than that of any of the solutes. The exosomes in the sample are then separated based on their size and mass instead of density. This allows the separation of exosomes with similar densities but different sizes.

### Example 8

Using a Nanomembrane Concentrator to Isolate Exosomes

A nanomembrane concentrator has a polyethersulfone membrane and a uniform pore size of 13 nm. Such a nanomembrane concentrator provides exosomes with less than 100 nm in diameter. After brief centrifugation to remove cells and cell debris, the clear supernatant containing exosomes are added to nanomembrane concentrator, and then centrifuged at 3,000×g at 20° C. for 10-30 min. The retentate is recovered, and the membrane is washed with solubilizing buffer, and combined with the retentate to increase the yield of exosomes.

### Example 9

Using a Syringe Filter-Based Fractionation to Isolate Exosomes

A syringe is equipped with two membranes in tandem configuration so that exosomes are captured on the lower membrane whereas larger extracellular vesicles are retained on the upper membrane when a sample is passed through the two membranes. The syringe is filled with exosomes-containing sample, and forced to pass through the filters. Exosomes, based on their sizes, are trapped between the two membranes.

### Example 10

Sequential Filtration of Exosomes

Samples containing exosomes, cells, and cell debris are filtered using 100 nanometer filter, depleting floating cells and larger debris from the sample. The filtered sample is subjected to a second filtration using hollow fibers with 500 kDa molecular weight cut-off. The retentate is then filtered with 100 nm track-etch filter whereby exosomes are recovered from the filtrate.

### Example 11

Size-Exclusion Chromatography

A size-exclusion column having a porous stationary phase is used to sort macromolecules and particulate matters according to their sizes. The instrument comprises a liquid chromatography system with a binary pump, an auto injector, a thermostatted column oven, and a UV-visible detector operated by the Class VP software (Shimadzu Corporation). The Chromatography columns are TSK Guard column SWXL, 6×40 mm, and TSK gel G4000 SWXL, 7.8×300 mm (Tosoh Corporation). Detectors, Dawn 8 (light scattering), Optilab (refractive index) and QELS (dynamic light scattering) are connected in series following the UV-visible detector. The eluent buffer is 20 mM phosphate buffer with 150 mM NaCl, pH 7.2. This buffer is filtered through a pore size of 0.1 m and degassed for 15 min before use. The chromatography system is equilibrated at a flow rate of 0.5 ml/minute until the signal in Dawn 8 is stabilized at around 0.3 detector voltage units. The UV-visible detector is set at 220 nm and the column's temperature is equilibrated to 25° C. Isocratic elution mode is run for 40 min. The volume of sample containing exosomes ranges from 50 to 100 μL. The % area of the exosome peak vs. all other peaks are integrated from the UV-visible detector. The hydrodynamic radius, Rh is computed by the QELS and Dawn 8 detectors. The highest count rate (Hz) at the peak apex is taken as the Rh. Peaks of the separated components visualized at 220 nm are collected.

### Example 12

Magnetic Separation of Exosomes

Antibodies against molecules at the surface of exosomes are utilized to capture exosomes. Anti-CD63 antibodies are conjugated with magnetic particles, added to the sample containing exosomes. The mixed sample is then placed in a tube. Upon placing the tube in a magnetic field, exosomes having CD63 on their surface are bound to the inside wall of the tube by the magnetic field. The sample is then repeated washed with phosphate buffered saline to remove unbound particulates. After the wash, the tube is detached from the magnets, and the washed exosomes are released.

### Example 13

Selective Precipitation of Exosomes

Polyethylene glycol (PEG) is used to isolate exosomes. A sample containing exosomes is pre-cleared by passing through a Sephadex G-25 column, removing subcellular particles such as lipoproteins. The cleared sample is then incubated in a solution containing PEG with a molecular weight of 8,000 Da. After incubation at 4° C. overnight, the precipitate containing exosomes is isolated by means of either low speed centrifugation or filtration.

### Example 14

Microfluidics Separation of Exosomes

Microfluidic devices having fabricated surfaces are designed to isolate exosomes based on their properties. Exosomes are isolated using an acoustic nanofilter, utilizing ultrasound standing waves to separate exosomes according to their size and density. An acoustic nanofilter moves larger particles faster toward a node at the end of a channel, as they experience stronger radiation forces. Alternatively, a porous silicon-based nanowire having a micropillar structure is fabricated on a surface. The micropillars are spaced in a lattice with fixed distances to trap exosomes with diameters less than 100 nm. The trapped exosomes are recovered by dissolving the porous silicon nanowires in phosphate buffered saline.

### Example 15

Isolating Botulinum Toxin Type B

Botulinum toxin type B is obtained by establishing and growing cultures of Clostridium botulinum in a fermenter and then harvesting and purifying the fermented mixture. Generally, botulinum toxin Type B is isolated as a complex from high titer fermentations of C. botulinum cultures, according to methods well known in the art. Stock cultures can be obtained in the United States by institutions holding a license from the Center for Disease Control (CDC) and elsewhere, according to the national regulations on distribution of the organism. For purification of botulinum toxin Type B, C. botulinum Okra or Bean B are appropriate starting materials. Frozen stock cultures are inoculated into test tubes containing culture medium such as thioglycollate medium or trypticase peptone medium, and cultures grown and processed. Briefly, cultures are expanded according to methods known in the art to produce sufficient amount of bacterial starting material to produce a desired yield of toxin. Generally, about 20 liters of bacterial culture is required to produce 0.5 grams of toxin. The culture is brought to room temperature, and the pH of the culture is adjusted to pH 3.5 with sulfuric acid or another suitable acid. The resulting precipitate is allowed to settle, and the cleared supernatant is decanted. Calcium chloride is then added to the precipitate with stirring and the volume is increased with deionized water, such that the final concentration of CaCl₂ is about 150 mM. The pH is raised to near neutrality (pH 6.5) and the toxin solution is clarified by centrifugation. The toxin is reprecipitated by adjustment of the pH to 3.7. The resulting precipitate is allowed to settle, and the toxic precipitate is collected by centrifugation, then re-dissolved in buffer (pH 5.5) and exhaustively dialyzed overnight against the same buffer. The dialyzed toxin is centrifuged and the resulting supernatant chromatographed through an anion exchange column (DEAE). The unbound fraction is collected and tested for protein content. Toxin complexes are precipitated from this fraction by addition of ammonium sulfate to about 60% saturation. The pellet is dissolved in phosphate buffer and dialyzed against the same buffer (pH 7.9). This purified toxin preparation can be used to prepare the formulation.

### Example 16

Preparing Botulinum Toxin Type A

Cultures of the bacteria Clostridium botulinum type A NCTC 2916 are grown up from a verified seed stock and inoculated into a 30 liter fermenter operated under anaerobic conditions, according to standard conditions known in the art. Toxin yield is monitored continuously (for example by LD₅₀ determination), and when maximum yield is achieved (roughly 2×10⁶ mouse LD₅₀/ml), the culture is acidified (adjusted with 3 N H₂SO₄ to pH 3.5, and the toxin is harvested by centrifugation. This precipitated crude toxin is re-dissolved and extracted with 0.2 M phosphate buffer (pH 6.0), followed by ribonuclease treatment (100 μg/ml at 34°) and precipitation using NH₄SO₄ (60% saturation at 25°). The precipitate is then resuspended and subjected to DEAE-Sephacel ion-exchange chromatography at pH 5.5 (following batch pre-adsorption). Fractions are monitored for activity, and active fractions are again precipitated using NH₄SO₄ (60% saturation at 25°). The precipitate can be stored and re-dissolved to make a formulation suitable to be added to exosomes.

### Example 17

Transient Expression of Botulinum Toxin Type A

To transiently express an active BoNT/A in a cell line, about 1.5×10⁵ SH-SY5Y cells are plated in a 35 mm tissue culture dish containing 3 mL of complete Dulbecco's Modified Eagle Media (DMEM), supplemented with 10% fetal bovine serum (FBS), 1× penicillin/streptomycin solution (Invitrogen) and 1× MEM non-essential amino acids solution (MEM) (Invitrogen, Inc, Carlsbad, CA), and are grown In a 37° C. incubator under 5% carbon dioxide until cells reach a density of about 5×10⁵ cells/mL (6-16 hours). A 500 μL transfection solution is prepared by adding 250 μL of OPTIMEM Reduced Serum Medium containing 15 μL of UpofectAmine 2000 (Invitrogen, Carlsbad, CA) incubated at room temperature for 5 minutes to 250 μL of OPTI-MEM Reduced Serum Medium containing BoNT/A expression plasmid. This transfection is incubated at room temperature for approximately 20 min. The complete, supplemented DMEM media is replaced with 2 mL of OPTI-MEM Reduced Serum Medium and the 500 μL transfection solution is added to the SH-SY5Y cells, and the cells are incubated In a 37° C. incubator under 5% carbon dioxide for approximately 6 to 18 hours. Transfection media is replaced with 3 mL of fresh complete, supplemented DMEM and the cells are incubated in a 37° C. incubator under 5% carbon dioxide for 48 hours.

### Example 18

Confirming the Transient Expression of BoNT/A

Cells are harvest by rinsing cells once with 3.0 mL of 100 mM phosphate-buffered saline, pH 7.4 and lysing cells with a buffer containing 50 mM N-(2hydroxyethyl) piperazine-N′(2-ethanesufonic acid) (HEPES), pH 6.8 150 mM sodium chloride, 1.5 mM magnesium chloride, 10% (v/v) glycerol, 1 mM ethylene glycol bis(j-aminoethyl ether) N, N, N, N′tetraacetic acid (EGTA), 2% (v/v) Triton-X 100 (4-octylphenol polyethoxylate) and 1× Complete protease inhibitor cocktail (Roche Applied Science). Cell samples are added to 2× LDS Sample Buffer and expression is measured by Western blot analysis.

### Example 19

Stable Expression of Botulinum Toxin Type A

To generate a stably-integrated cell line expressing BoNT/A, approximately 1.5×10⁵ SH-SY5Y cells are plated in a 35 mm tissue culture dish containing 3 ml of complete DMEM, supplemented with 10% FBS, 1× penicillin/streptomycin solution and 1× MEM nonessential amino acids solution, and grown in a 37° C. incubator under 5% carbon dioxide until cells reach a density of about 5×10⁵ cells/mI (6-16 hours). A 500 μL transfection solution is prepared by adding 250 μL of OPTI-MEM Reduced Serum Medium containing 15 μL of LipofectAmine 2000 (Invitrogen) incubated at room temperature for 5 min to 250 μL of OPTI-MEM Reduced Serum Medium containing 5 ug of a plasmid containing BoNT/A and a selection marker for stable transfection. This transfection is incubated at room temperature for approximately 20 min. The complete, supplemented DMEM media is replaced with 2 mL of OPTI-MEM Reduced Serum Medium and the 500 μL transfection solution is added to the SH-SY5Y cells and the cells are incubated in a 37° C. incubator under 5% carbon dioxide for approximately 6 to 18 hours. Transfection media is replaced with 3 ml of fresh complete, supplemented DMEM and the cells are incubated in a 37° C. incubator under 5% carbon dioxide for approximately 48 hours. Media is replaced with 3 ml of fresh complete DMEM, containing 5 ug/mL blasticidin, 10% FBS, 1× penicillin/streptomycin solution (Invitrogen) and 1× MEM non-essential amino acids solution (Invitrogen). Cells are incubated in a 37° C. incubator under 5% carbon dioxide for approximately 3-4 weeks, with old media being replaced with fresh blasticidin selective, complete, supplemented DMEM every 4 to 5 days. Once blasticidin-resistant colonies are established, resistant clones are replated to new 35 mm culture plates containing fresh complete DMEM, supplemented with approximately 5 ug/mL of blasticidin, 10% FBS, 1× penicillin/streptomycin solution (Invitrogen, Inc) and 1× MEM non-essential amino acids solution (Invitrogen), until these cells reach a density of 6 to 20×10⁵ cells/mL.

### Example 20

Confirming the Stable Expression of Botulinum Toxin Type A

To test for expression of BoNT/A from SH-SY5Y cell lines that have stably-integrated a cDNA for BoNT/A, approximately 1.5×10⁵ SH-SY5Y cells from each cell line are plated in a 35 mm tissue culture dish containing 3 mL of blasticidin selective, complete, supplemented DMEM and grown in a 37° C. incubator under 5% carbon dioxide until cells reach a density of about 5×10⁵ cells/ml (6-16 hours). Media is replaced with 3 mL of fresh blasticidin selective, complete, supplemented DMEM and cells are incubated in a 37° C. incubator under 5% carbon dioxide for 48 hours. Both media and cells are collected for expression analysis of BoNT/A. Media is harvested by transferring the media to 15 mL snap-cap tubes and centrifuging tubes at 500× g for 5 min to remove debris. Cells are harvest by rinsing cells once with 3.0 mL of 100 mM phosphate-buffered saline, pH 7.4 and lysing cells with a buffer containing 62.6 mM 2-amino-2-hydroxymethyl-1,3-propanediol hydrochloric acid (Tris-HCl), pH 6.8 and 2% sodium lauryl sulfate (SDS). Both media and cell samples are added to 2× LDS Sample Buffer (Invitrogen) and expression is measured by Western blot analysis using anti-BoNT/A antibodies. The established SH-SY5Y cell line showing the highest expression level of BoNT/A is selected for large-scale expression using 3 L flasks. Procedures for large-scale expression are as outlined above except the starting volume is approximately 800-1000 mL of complete DMEM and concentrations of all reagents are proportionally increased for this volume.

### Example 21

Encapsulation by Sonication

Cargo molecules are mixed with about 10¹¹ exosomes in 1 ml phosphate-buffered saline. The mixture is incubated at 37° C. for 1 hour with shaking. The mixture is then sonicated at 20% amplitude, 6 cycles of 30 seconds on and off for three minutes with a two-minute cooling period between each cycle. After the sonication, the mixture is incubated at 37° C. for 1 hour to allow for recovery of the exosomes.

### Example 22

Encapsulation by Extrusion

Exosomes are prepared at a concentration of 0.15 mg/ml of total protein. The cargo protein is prepared in PBS at a concentration of 0.5 mg/ml. The exosomes and the cargo protein are mixed at a ratio about 4:6, and then extruded 10 times through a commercial extruder, such as Avanti Lipids extruder, with 200 nm-pores diameter. The loaded exosomes are purified by gel-filtration chromatography with Sepharose column.

### Example 23

Encapsulation Using a Permeabilizer

Exosomes are prepared at a concentration of 0.15 mg/ml of total protein. The cargo protein is prepared in PBS at a concentration of 0.5 mg/ml. The exosomes and the cargo protein are mixed at a ratio about 4:6, and incubated for 20 min in the presence of 0.2% saponin.

### Example 24

Encapsulation by Transfection

1 ug of the DNA is mixed with 1 μL of the transfection reagent, and 3 ug of exosomes in 100 μL of DMEM. The solution is incubated for 3 hours at 37° C. To confirm the transfection of the nucleic acid into the exosomes, the transfected mixture is treated with DNase or RNase to digest unincorporated nucleic acid, and subjected to a conventional gel electrophoresis assay to determine the presence of undigested nucleic acid in the sample.

### Example 25

Encapsulation by Electroporation

As an example, electroporation is carried out using a voltage in the range of 150 mV to 250 mV, particularly 200 mV. In another embodiment, electroporation is carried out with capacitance between 25 uT and 250 uT, particularly between 25 uT and 125 uT.

### Example 26

Investigating Exosome's Skin Permeability

About 40 μL of the exosomes are applied per square centimeter on the surface of donated human skin. The skin is allowed to dry prior to be embedded for cryosection. The slices are prepared by covering the skin samples with a thin layer of Tissue-Tek (Sakura), and immersed for 60 seconds into 2-methylbutane cooled by liquid nitrogen. The samples are then stored at −80° C. The samples are cut perpendicular to the surface into 30 um slices using a Crypotome (Thermo Scientific), and transferred to a glass pre-treated with poly-L-Lysine. Raster image correlation spectroscopy (RICS) is used to detect the fluorescent signal of the sliced samples. The microscopy is set up as follows: a Ti:Sa laser is used for the excitation wavelength of 838 nm. The fluorescent signals is collected using bandpass filters ET Bandpass 572/35 nm and BrighLine HC 676/29 nm. The light is divided by 620-DCXXR beam splitter. The detector is Hamamatsu H7422P-40 photomultiplier tube. About 50-100 images are combined for RICS measurements. In addition, stimulated emission depletion (STED) microscopy is used to visualize the layers of skin, such as stratum corneum, in the order of 100 nm. To visualize the skin layers, an atto-488-DPPE dye is used with the excitation wavelength of 488 nm and the depletion wavelength of 592 nm.

### Example 27

Encapsulation by Receptor-Mediated Endocytosis

Cells of neuronal lineage expressing synaptic vesicle 2 proteins are cultured. Exosomes secreted from the cell culture are harvested and isolated by ultrafiltration. About 10¹¹ exosomes are prepared in 1 ml of cell culture media, and mixed with cargo molecules. The mixture is incubated at 37° C. for 12 hours in a humidified CO₂ incubator. The degree of encapsulation is then examined by standard antibody-mediated fluorescent microscopy technique. To do so, the mixture is fixed with formaldehyde and the exosome are permeated with a mild detergent, such as Triton X or NP40, and labeled with a fluorescent antibody against the cargo molecule. The exosome is also counterstained with a fluorescent antibody against lipid molecules in the membrane. The number of exosomes containing the cargo molecule is counted. Based on the result, the amount of cargo molecule is adjusted until 95% or more of the exosomes encapsulate the cargo molecule.

### Example 28

Encapsulation of Catalytic Domain of the Neurotoxin

Following the method described in EXAMPLE 17, a plasmid containing the light chain of BoNT/A is used instead of a full-length cDNA of BoNT/A.

### Example 29

Expression of BoNTs in Bacterial Cells

The cDNA of BoNT/A is cloned in the pET32a expression vector. Recombinant molecules are expressed in E. coli in selective media using 1 mM isopropyl-β-D-thiogalactopyranoside at 16° C. for 20 hours. Cells are harvested and lysed, and the lysate is clarified before purification of target protein by fast protein liquid chromatography with BuHP Sepharose (GE Healthcare Life Sciences) and Q Sepharose High Performance (GE Healthcare Life Sciences). The partially purified molecule is then cleaved with Lys-C to yield the active polypeptide, which is polished down a phenyl Sepharose High Performance (GE Healthcare Life Sciences) column. The purity is determined by densitometry, and molecule identity is confirmed by SDS-polyacrylamide gel electrophoresis (PAGE) and Western blot analysis.

### Example 30

Expression of BoNTs in Insect Cells

DNA encoding cDNA of BoNT/A is synthesized de novo, and optimized for expression in Sf9 cells. By substituting two amino acid (E₂₂₄>A and Y₃₆₆>A), which have been previously shown to reduce the toxicity of rBoNT derivatives by 100,000-fold compared to wt BoNT/A. A specific protease cleavage site is placed between the affinity tags at the N- and C-termini, and between the light chain and heavy chain domains of the native, wild-type BoNT/A sequence. Following standard transfection of the cDNA and Sf9 cell culture, rBoNT/A is recovered from the culture medium as a soluble, stable, disulfide-bonded pro-peptide. The single chain pro-peptide is purified using a 2-step tandem affinity purification procedure with Ni²⁺-NTA agarose and strep-tactin-agarose, utilizing a strep-tactin II tag on the C-terminus and a poly-histidine tag on the N-terminus. After purification to homogeneity, the pro-peptide is processed with TEV protease to yield a heterodimer, and the activating protease is removed by affinity chromatography on Ni²⁺-NTA agarose to yield the final active polypeptide, which is polished using gel-filtration chromatography. The final polypeptide is sterilized by filtration through 0.22-micron filters. Purity is analyzed by SDS-PAGE under reducing and non-reducing conditions.

The invention claimed is:

1. A composition comprising,
1) Exosomes derived from exosome-producing cells; and
2) Neurotoxins selected from the group consisting of botulinum toxin A, B, C1, D, E, F, and G, and tetanus toxin, wherein said neurotoxins are encapsulated in said exosomes and said neurotoxins are non-full-length polypeptide containing the catalytic domain of said neurotoxins that is unable to be bound to said neurotoxins' natural receptors.

2. The composition of claim 1, wherein said exosomes contain neuronal cell specific glycoprotein.

3. The composition of claim 1, wherein said exosomes contain Synapsin I.

4. The composition of claim 1, wherein said exosome-producing cells are neuronal cells, neuronal stem cells, neuronal stem cells derived from an induced pluripotent stem cell, or neuronal cells differentiated from an induced pluripotent stem cell.

5. The composition of claim 1, wherein said exosome-producing cells are fibroblasts.

6. The composition of claim 1, wherein said exosome-producing cells are human dermal microvascular endothelial cells.

7. The composition of claim 1, wherein equal to or less than 30 percent of said exosomes's lumen is filled with said neurotoxins.

8. The composition of claim 1, wherein said exosomes have a diameter of equal to or less than about 150 nm.

9. The composition of claim 1, wherein said exosomes have a diameter of equal to or less than about 40 nm.

10. The composition of claim 1, wherein said neurotoxins are derived from bacterial, yeast, insect, or mammalian cells transiently or stably expressing said neurotoxins from the cDNAs of said neurotoxins.

11. The composition of claim 1, wherein said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

12. The composition of claim 1, wherein said composition further comprise an emollient selected from the group consisting of cerotyl linoleate, diisoestearyl dimerate, diisopropyl dimerate, stearyl oleate, stearyl linoleate, cerotyl oleate, melissyl oleate, melissyl linoleate, and cerotyl eicosapentanoate.

13. The composition of claim 1, wherein said composition further comprise water, disodium EDTA, soldium hyaluronate, glycerin, palmitoyl tripeptide, dextran, caproyl tetrapeptide, xanthan gum, cetostearyl alcohol, shea butter, jojoba oil, coconut oil, cetearyl ethylhexanoate, squalene, carbomer, pheoxyethanol, ethylhexylglcerin, NaOH, corundum powder, Asparagopsis armata extract, collagen, magnesium stearate, cellulose, lactose, mannitol, methyl cellulose, or a combination thereof.

14. The composition of claim 1, wherein said composition further comprise a buffered solution selected from the group consisting of phosphate-buffered saline, Hanks balance salt solution, or saline.

15. The composition of claim 1, wherein said composition is packaged in a roller, a ball-pointed liquid container, a liquid dispenser, a sprayer, a spatula, a dropper, a hand-operated pump, or a tube.

16. The composition of claim 1, wherein the amount of said exosomes in a package is about 4 mg.

17. A method of preparing exosomes comprising,
1) Culturing induced pluripotent stem cells;
2) Differentiating said induced pluripotent stem cells to neuronal stem cells; and
3) Isolating exosomes, wherein said isolating is ultracentrifugation, ultrafiltration, size-exclusion chromatography, immunoisolations, precipitation, or microfluidics-based isolation.

18. A method of preparing exosomes comprising, culturing exosome-producing cells and encapsulating neurotoxins, wherein said exosome-producing cells are 1OT1/2, BALB/3T3, L-M, NB4, 1A3, NIE-1 15, NG108-15, NIH3T3, NCTC, Neuro-2A, PC12, GH1, GH3, C6, L2, CHO, OHO, 6E6, PK15, LLC-PK1, ST, ESK-4, CPAE, BT, FB2, SBAC, NBL-6, COS-1, COS-7, or VV-1, SH-SY5Y, SK-N-DZ, SK—N—Fl, SK—N—SH, BE(2)-C, HeLa, HEK 293, MCF-7, HepG2, HL-60, IMR-32, SW-13, OHP3, or CHPS; and wherein said encapsulating is receptor-mediated endocytosis, passive diffusion, sonication, saponication, heating, emulsification, freezing and thawing, or use of solvents.

19. A method of applying a neurotoxin to skin, comprising:
1) preparing said skin by a method selected from the group consisting of cleaning, surfactant treatment, derma abrasion, exfoliation, chemical peel, laser resurfacing, hair removal, massaging, warming, and cleaning of the skin; and
2) Applying a composition comprising exosomes having a diameter of equal to or less than 40 nm, and neurotoxins.

20. The method of claim 19, wherein said surfactant is sodium lauryl sulfate, ammonium lauryl sulfate, disodium lauryl sulfosuccinate, cocoamphocarboxyglycinate, cocoamidopropyl betaine, or alpha-olefin sulfonate.

21. The method of claim 19, wherein said exosomes have neuronal cell specific glycoprotein.

22. The method of claim 19, wherein said exosomes have Synapsin I.

23. The method of claim 19, wherein said neurotoxins are selected from the group consisting of botulinum toxin A, B, C1, D, E, F, and G, and tetanus toxin wherein said neurotoxins are encapsulated in said exosomes and said neurotoxins are non-full-length polypeptide containing the catalytic domain of said neurotoxins that is unable to be bound to said neurotoxins' natural receptors.

24. The method of claim 19, wherein said exosomes are derived from neuronal cells, neuronal stem cells, neuronal stem cells derived from an induced pluripotent stem cell, or neuronal cells differentiated from an induced pluripotent stem cell.

25. The method of claim 19, wherein said exosomes are derived from fibroblasts.

26. The method of claim 19, wherein said exosomes are derived from human dermal microvascular endothelial cells.

27. The method of claim 19, wherein said compositions are delivered to neuronal cells.

28. The method of claim 19, wherein said compositions are delivered to human fibroblasts.

29. The method of claim 19, wherein said compositions are delivered to endothelial cells.

30. The method of claim 19, wherein equal to or less than 30 percent of the lumen of said exosomes are filled with said neurotoxins.

31. The method of claim 19, wherein said exosomes further comprise a cargo molecule selected from the group consisting of codeine, fentanyl, hydrocodone, hydromorphone, meperidine, morphine, oxycodone, tramadol, alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydro morphine, dimenoxadol, dimepheptanol, dimethylthiambutene, dioxaphetylbutyrate, dipipanone, eptazocine, ethoheptazine, ethylmethylthiambutene, ethylmorphine, etonitazene, heroin, hydroxypethidine, isomethadone, ketobemidone, levallorphan, levorphanol, levophen-acylmorphan, lofentanil, meptazinol, metazocine, methadone, metopon, myrophine, nalbuphine, narceine, nicomorphine, norlevorphanol, normethadone, nalorphine, normorphine, norpipanone, opium, oxymorphone, papaveretum, pentazocine, phenadoxone, phenomorphan, phenazocine, phenoperidine, piminodine, piritramide, propheptazine, promedol, properidine, propiram, propoxyphene, sufentanil, and tilidine.

32. The method of claim 19, wherein said composition further comprise an emollient selected from the group consisting of cerotyl linoleate, diisoestearyl dimerate, diisopropyl dimerate, stearyl oleate, stearyl linoleate, cerotyl oleate, melissyl oleate, melissyl linoleate, and cerotyl eicosapentanoate.

33. The method of claim 19, wherein said composition further comprise water, disodium EDTA, soldium hyaluronate, glycerin, palmitoyl tripeptide, dextran, caproyl tetrapeptide, xanthan gum, cetostearyl alcohol, shea butter, jojoba oil, coconut oil, cetearyl ethylhexanoate, squalene, carbomer, pheoxyethanol, ethylhexylglcerin, NaOH, corundum powder, Asparagopsis armata extract, collagen, magnesium stearate, cellulose, lactose, mannitol, methyl cellulose, or a combination thereof.

34. The method of claim 19, wherein said composition further comprise a buffered solution selected from the group consisting of phosphate-buffered saline, Hanks balance salt solution, or saline.

35. The method of claim 19, wherein said composition is packaged in a roller, a ball-pointed liquid container, a liquid dispenser, a sprayer, a spatula, a dropper, a hand-operated pump, or a tube.

36. The method of claim 19, wherein the amount of said exosomes in a package is about 4 mg.

37. A method of selecting exosomes suitable for releasing neurotoxins to neuronal cells in human skin comprising:
1) Culturing exosome-secreting cells;
2) Modifying said exosome-secreting cells or exosomes secreted therefrom to carry one or more neurotoxins in said exosomes;
3) Culturing neuronal cells in the presence of said exosomes;
4) Selecting for exosomes of which said neurotoxins are found in said neuronal cells;
5) Optionally sorting said exosomes of step four in accordance to their sizes;
6) Applying said selected and optionally sorted exosomes to a donated human skin; and
7) Selecting for exosomes of which said neurotoxins are found in the dermis layer of said human skin.

38. The method of claim 37, wherein said culturing is a primary culture of cells derived from peripheral neuron.

39. The method of claim 37, wherein said modifying is selected from the group consisting of transfection, transduction, extrusion, sonication, diffusion, saponication, heating, emulsification, and freezing and thawing.

40. The method of claim 37, wherein said exosomes are isolated from said exosome-producing cells by a method selected from the group consisting of ultracentrifugation, ultrafiltration, size-exclusion chromatography, immunoisolations, precipitation, and microfluidics-based isolation.

41. The method of claim 37, wherein said sorting is selected from the group consisting of ultracentrifugation, ultrafiltration, size-exclusion chromatography, immunoisolations, precipitation, and microfluidics-based isolation.

42. The method of claim 37, wherein said applying is incubating said skin with said exosomes for 2, 4, 8, 12, or 24 hours.

43. The method of claim 37, wherein said selecting of step seven is performed by a method selected from the group consisting of immunostaining, fluorescent microscopy, and western blot.
